(19) United States
(12) Patent Application Publication
(10) Pub. No.: US 2026/0063609 A1
(43) Pub. Date: Mar. 5, 2026

(54) PROCESS FOR DETERMINING PHOSPHONATE IN SALINE WATERS

(71) Applicants: Petróleo Brasileiro S.A. – Petrobras, Rio de Janeiro (BR); Universidade Federal do Rio de Janeiro — UFRJ, Rio de Janeiro (BR)

(72) Inventors: Marco Antônio Gomes TEIXEIRA, Niterói (BR); Renato Malbar Musiello BARCELLOS, Rio de Janeiro (BR); Aline Machado DE AZEVEDO NOVAES, Rio de Janeiro (BR); Rogerio Mesquita DE CARVALHO, Rio de Janeiro (BR); Rafaella Magliano Balbi DE FARIA, Rio de Janeiro (BR); Renata Recker SOUSA DE SÁ, Rio de Janeiro (BR); Caroline Dos Santos SILVA, Rio de Janeiro (BR); Amanda Ferreira DA SILVA, Rio de Janeiro (BR); Ana MEHL, Rio de Janeiro (BR); Mariana Dias MARTINS, Rio de Janeiro (BR); Helida Vasques Peixoto VIEIRA, Rio de Janeiro (BR)

(21) Appl. No.: 19/273,873

(22) Filed: Jul. 18, 2025

(30) Foreign Application Priority Data
Aug. 27, 2024 (BR) 1020240175581

(51) Int. Cl. G01N 33/18

(52) U.S. Cl. CPC G01N 33/182

(57) ABSTRACT

The present disclosure discloses a process that determines the presence of phosphonate in saline water without specific equipment and can be used with simple resources if necessary. The process is carried out in two parts: 1) conversion of phosphonate by thermal method, and 2) determination of phosphate applying the analytical method based on digital images (DIA). Furthermore, the process for detecting phosphonates was developed with the concept of a portable analytical method capable of being performed even by analysts without training in chemical operations.

### CROSS-REFERENCE TO RELATED APPLICATIONS

This application claims priority to Brazilian patent application Ser. No. 1020240175581, filed Aug. 27, 2024, which is incorporated herein in its entirety by reference hereto.

### FIELD OF APPLICATION

The present disclosure discloses a process for detecting residual phosphonates in waters where oil extraction is carried out, which use antiscalant with phosphonates in the composition to control saline incrustations, including on board offshore production facilities.

### BACKGROUND

Phosphonates are used on a large scale as antiscalant in several applications, including for oil formation waters. They prevent the deposition of salts, including during oil production, thus avoiding problems with clogging in the lines and equipment of the processes in which they are used. Detecting the presence and efficiently controlling the concentration of these compounds so that it does not fall below the ideal for antiscalant action and, similarly, so that it is not added in excess in nature, is a technological solution that offers advantages in these processes.

Phosphonate control generally involves (usually after some reaction) the application of sophisticated, high-cost equipment such as ion chromatography. Based on chromatographic separation, several detection methods have been developed, such as indirect photometric detection and mass spectrometric detection (Shamsi S. A., Danielson N. D., Ion chromatography of polyphosphates and polycarboxylates using a naphthalenetrisulfonate eluent with indirect photometric and conductivity detection, J. Chromatogr. A 653 (1993) 153 to 160; Nowack B., Determination of phosphonates in natural waters by ion-pair high performance liquid chromatography, J. Chromatogr. A 773 (1997) 139 to 146; Carsten K. Schmidt, Brigitte Raue, Heinz-Jürgen Brauch & Frank Sacher, Trace-level analysis of phosphonates. Environmental waters by ion chromatography and inductively coupled plasma mass spectrometry. Intern. J. Environ. Anal. Chem., 2014 Volume 94, Number 4, 385 to 398). Indirect detection methods combined with capillary electrophoresis are also used (Shamsi S. A., Danielson N. D., Ribonucleotide electrolytes for capillary electrophoresis of polyphosphates and polyphosphonates with indirect photometric detection, Anal. Chem. 67 (1995) 1845 to 1852). All of these methodologies require sample collection and transportation to laboratories that have the sophisticated techniques they use. Particularly in the case of oil production in offshore environments and other processes with remote characteristics, this is a possibility that does not occur for detection on board production facilities.

A simpler protocol would be ideal because it is easy to perform in the field. It would then be necessary to perform a phosphonate reaction that would promote a method of easy subsequent detection. Possibly a specific color formation, for example, by direct reaction of the phosphonate present in saline matrices, but such a reaction is not known.

However, since the presence of precursor groups of phosphate anions is the main chemical characteristic to be perceived in the evaluation of the chemical structure of phosphonates, it is a development found in the literature of methodologies that act by transforming them into orthophosphate, through a conversion process. Phosphate is an anion whose concentration in several aqueous media is of interest, including for the evaluation of natural waters, for example, so it is possible to use methods for this anion and, once the conversion of phosphonates into phosphate is carried out, methodologies with varied principles can be carried out, including the generation of color, using different reactions already shown in the literature (for example, in SM-4500-P, precisely for natural waters). Color generation has characteristics of methodologies that can be easily adapted for use in the field. Therefore, the question of conversion remains.

The literature shows types of phosphonate conversion with the use of oxidants that, together with catalysts, break down the phosphonate molecules, transforming them into orthophosphate (Rott E, Minke, Bali U, Steinmetz H. Removal of phosphonates from industrial wastewater with UV/Fell, Fenton and UV/Fenton treatment. Water Research 122 (2017) 345e354; Assalim M R., Moraes S G., Queiroz S C. N., Ferracini V. L and Duran N. Studies on degradation of glyphosate by several oxidative chemical processes: Ozonation, photolysis and heterogeneous photocatalysis. Journal of Environmental Science and Health Part B (2010) 45, 89 to 94). The trend observed from the literature review is towards methods that apply advanced oxidation processes (AOP), in which the oxidation accelerator is ultraviolet light that promotes the breakdown of the complex molecule into orthophosphate (Zhanga X, Guoa W, Zhenga Y, Chengb Z, Qib X, and Gaoa C. Rapid determination of aminotris(methylenephosphonic acid) in water by ultraviolet photooxidation. Instrumentation science and technology, 2017, volume 45, number 4, 459 to 468; Wang Z, Chen G, Patton S, Ren C., Liu J, Liu H. Degradation of nitrilotrismethylenephosphonic acid (NTMP) antiscalant via persulfate photolysis: Implications on desalination concentrate treatment. WaterResearch 159 (2019) 30 and 37). Some systems optimized for this make the reaction reasonably fast and can be considered for field application. An example of this, used for comparison with the present disclosure, was the method of the company HACH. In this, a commercially available technology is used for conversion with POA and completion by detecting phosphate using visible spectrophotometry.

However, the basis of the HACH methodology (or any other based on POA) does not allow for use in unexpected problems that occur on board, as it is not based on simple resources. Complex reagents are used, which need to be packaged in specific sachets, specific lamps and non-conventional analytical equipment, which must be taken to an offshore environment and used specifically for the methodology. Additionally, there are limitations associated with components present in the matrix that can interfere with the conversion process and the analysis itself, such as chloride. Values above 5000 mg/L are already considered as interfering, significantly impacting the analysis and result.

The scientific article “Evaluation of Phosphate Level in Water Samples (Ogbomoso Rivers) Using UV-Visible Spectrophotometric Method” (Oladeji S., Adelowo F., Odelade K. Evaluation of Phosphate Level in Water Samples (Ogbomoso Rivers) Using UV-Visible Spectrophotometric Method. International Journal of Scientific Research in Environmental Sciences, (2016) 4 (4), 0102 to 0108) refers to a study that aims to evaluate the phosphate concentrations in some selected rivers in Ogbomoso in Oyo State, Nigeria, and compare the concentration with the recommended value. The major pollutant in the rivers of Ogbomoso is phosphate. The phosphate concentration in the water samples (expressed in mg per liter) was determined using a simple analytical method and UV-Visible spectrophotometric. The analysis was determined by the blue molybdenum phosphorus method in conjunction with a UV-Visible spectrophotometer. The phosphomolybdate complex was formed by reduction with hydrazine sulfate and addition of molybdate. The analysis in question follows the Lambert-Beer law at 860 nm in the concentration range of 0.5 to 3.5 ppm. The reagents used were hydrazine sulfate, potassium hydrogen phosphate and ammonium molybdate. However, the approach of the article could not be used in produced water samples considering the color of the solution (presence of oil, turbidity), the high levels of sodium, chloride and calcium, the presence of sulfide and high buffering capacity of the matrix. All these conditions are present in produced water samples, being a limitation to the application of the described procedure. The technical problem solved by the present disclosure is the determination of phosphonates in aqueous matrices from oil exploration and production activities, and not in aqueous matrices from rivers with phosphate concentration, the source of which is the use of fertilizers. In addition, the present disclosure performs the thermal conversion of phosphonate to phosphate, and makes use of digital image analysis, different from the concepts of spectroscopy.

The patent document BR 102021023976-0 discloses a portable analysis system that, based on a set of analytical methodologies based on digital images acquired by portable devices and accessories developed in an expeditious manner, enables the performance of the analytical test without technical in-depth knowledge by the analyzer. Such methodologies allow the evaluation of chemical species relevant to the Oil and Gas sector, for monitoring compositional parameters in aqueous samples and analytical monitoring of production and effluent treatment. In this way, rapid implementation is possible, considering possible diagnoses associated with the parameters of interest and monitoring of several processes. The application can be carried out in any laboratory present in an operational unit, whether offshore or onshore, and can also be used for direct field measurement, depending on the necessary environmental and operating conditions.

The Brazilian document BR 102021023976-0 refers to the concept of analysis that is the use of digital images, considering the induction of a response from the sample. In the case of alkalinity, it is the change in color, due to the reaction of an indicator in an acidic medium, sulfate and chloride are related to the induction of precipitation considering the turbidity response (and change in color). The reactions to induce the response required a specific study of the matrix, considering reaction interferents and the reaction itself with the analyte of interest. However, the document does not teach the thermal conversion of phosphonate to phosphate, nor does it perform the induction of a response from the reaction of phosphate with a solution to generate a response.

The patent document DE 2728706 refers to a procedure for the automatic determination of phosphate, in which, in order to perform the analysis in wastewater in such a way that automatic dosing without problems with precipitant is carried out according to the phosphate concentration of the wastewater, the molybdate-vanadate reagent solution is mixed with chloride ions. This prevents the formation of crystallites in the parts of the equipment through which the reagent solution flows, and the reproducible determination of phosphate becomes possible. The present disclosure, on the other hand, performs the thermal conversion of phosphonate to phosphate, and subsequent quantification of phosphate by inducing a response from the reaction of phosphate with a solution for generating a response.

As observed, the state of the art would benefit from a process for determining phosphonate in saline waters, which involves two main steps: the conversion of phosphonates to phosphate, without elaborate equipment, and the determination of phosphate applying the analytical method based on digital images. Furthermore, the present disclosure was developed for detecting residual phosphonates in field waters and for studies that are not covered by inventions from the state of the art, such as successfully distinguishing between PO₄⁻³ and phosphonates. Both are applicable to determining the active material content in product batches on board.

### BRIEF DESCRIPTION

A process for thermally converting phosphonates into phosphate without sophisticated equipment to subsequently quantify the phosphate using an analytical method based on digital images. More specifically, the process comprises the following steps:
(i) preparation of a sample containing phosphonate in a saline matrix with a NaCl concentration of 5 mol·L⁻¹;
(ii) a muffle with internal dimensions of 12 cm W×9.5 cm H×15 cm D was used to convert the phosphonate into phosphate;
(iii) a volume of 2 ml of the solution containing the phosphonate was transferred to a crucible with a lid, in duplicate;
(iv) the half-open crucible was placed in the muffle;
(v) the muffle was heated at a rate of 10° C. min⁻¹ to a temperature of 450° C. for 30 minutes;
(vi) transfer of the closed crucible to the desiccator;
(vii) with the crucible cold, the conversion residue was taken up with 5 ml of pure water;
(viii) determination of phosphate by an analytical method based on digital images.

### BRIEF DESCRIPTION OF THE FIGURES

The subject matter of this disclosure will become completely clear in its technical aspects from the detailed description that will be made based on the figures listed below, in which:

FIG. 1: Procedure for conversion on a hot plate: (a) elimination of water (30 minutes) (b) conversion of the phosphonate (1 hour);

FIG. 2: Crucible after conversion and aluminum foil improvising a lid;

FIG. 3: Steps for thermal conversion of the phosphonate in a muffle (a) sampling (b) placing the crucible in the muffle with the lid slightly open (c) removing the muffle (d) waiting for the crucible to cool;

FIG. 4: molecular formula of the antiscalant agent Dimethyl Triaminepentamethylene Phosphonic Acid (DTPMP).

### DETAILED DESCRIPTION

The present disclosure relates to a thermal conversion process with high temperature (450° C.) in a normal, ambient atmosphere. This temperature completely degrades organic matter in general, so that the phosphonate is transformed into orthophosphate. And to be used, this technique requires only a simple heating device.

The process comprises the following steps:
i. preparation of a sample containing phosphonate in a saline matrix with a NaCl concentration equal to 5 mol·L⁻¹;
ii. conversion of the phosphonate into phosphate, a muffle with internal dimensions of 12 cm W×9.5 cm H×15 cm D was used;
iii. a volume of 2 ml of the solution containing the quantity of phosphonate was transferred to a crucible with a lid, in duplicate;
iv. the half-open crucible was placed in the muffle;
v. the muffle was heated at a rate of 10° C. min⁻¹ to a temperature of 450° C. for 30 minutes;
vi. transfer of the closed crucible to the desiccator;
vii. using a cold crucible, the conversion residue is mixed with 5 ml of pure water;
viii. determination of phosphate by an analytical method based on digital images (reaction with ammonium molybdate in hydrochloric acid and stannous chloride resulting in the formation and reduction of phosphomolybdic acid, which is blue in color).

The process consisted of analyzing a solution prepared from a commercial phosphonate. It was chosen to analyze a solution already in the matrix with the greatest interference potential, that is, a saline matrix with a NaCl concentration equal to 5 mol·L⁻¹.

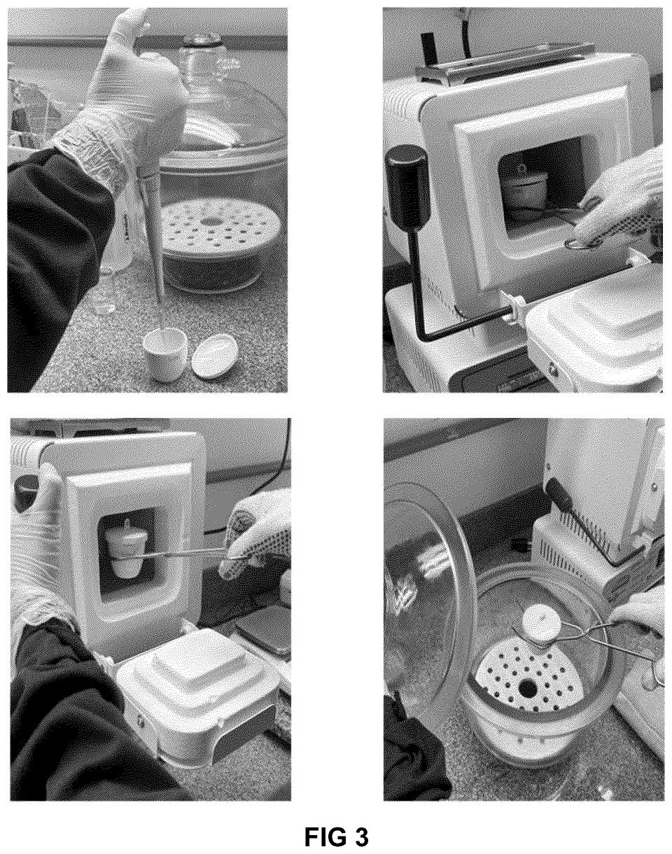

For the conversion of phosphonate to phosphate, a muffle with the following internal dimensions was used: 12 cm W×9.5 cm H×15 cm D. A volume of 2 mL of the solution containing a known quantity of phosphonate was transferred to a 50 mL porcelain crucible with a lid (in duplicate) and placed in the half-open muffle to allow steam and gases to escape during the conversion. The muffle was heated at a rate of 10° C./min⁻¹ to 450° C. and kept at this temperature for 30 minutes. After this time, the crucible was closed and removed from the muffle using long stainless steel tweezers and a glove specifically designed for working at high temperatures. For safety reasons, the closed crucible was placed in a desiccator and only when cold was the residue from the conversion taken up with 5 mL of pure water. FIG. 3 shows in detail the simple and easy-to-perform steps obtained for the process adapted for the thermal conversion of phosphonate.

The first tests were performed at 350° C., as this was considered a temperature range that would be more easily obtained on several commercial heating plates. However, as the phosphonate conversion was low, it was decided to perform the test at 450° C. After the thermal degradation concept had been implemented, a thermogravimetry curve was made in synthetic air of a commercial sample containing Polyamino Polyether Methylene Phosphonic Acid (PAPEMP—a widely used commercial phosphonate) as the main component, to evaluate the possibilities of the proposal. In fact, this curve indicated that at 450° C. there was no longer any oxidation process.

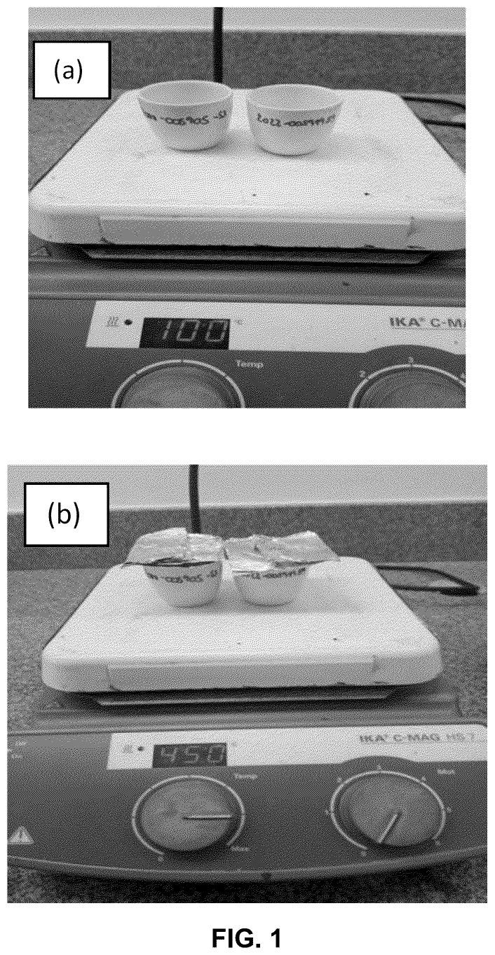

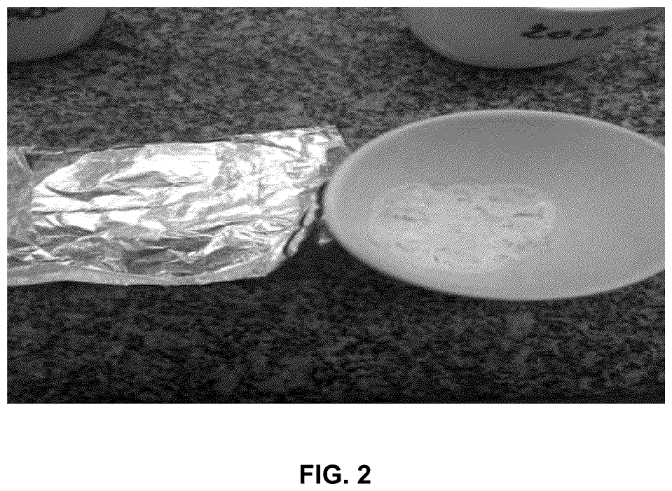

During the tests, the process consisted of preparing a solution from a commercial phosphonate in the matrix with the greatest interference potential, that is, a saline matrix (NaCl 5 mol·L⁻¹) that, if fully converted, would lead to 9 ppm of phosphorus (27.5 ppm of phosphate). For conversion on a hot plate at 450° C., 2 mL of the prepared solution were transferred to a 25 mL porcelain crucible (in duplicate). During the procedure, the water present was evaporated at 100° C. (30 minutes) and then the temperature was raised to 450° C. for 1 hour (FIG. 1). At the end of the conversion, the phosphate generated and other salts that existed in the matrix remained in the crucible, whereas in the first tests it was only NaCl (FIG. 2).

In FIG. 1, the aluminum foil that was added in the experiments to receive any projection was visibly splashed with salt from the solution, which indicated sample losses during the process.

The remaining solid was then taken up with 5 mL of deionized water measured with a 5 mL capacity micropipette. After a few tests, it was decided to abandon the hot plate process. Several factors led to this decision:
(a) low recovery and low repeatability in measurements, which were attributed to non-uniform heating of the crucible and projection during the process; the bottom of the crucible is heated while the rest of the crucible remains at a different temperature; and
(b) after evaporation of the water at 100° C., the salt layer at the bottom of the crucible (FIG. 1) causes projection of the sample as the temperature increases; attempts were made to use aluminum foil to prevent projection of the sample out of the crucible but were unsuccessful.

### Thermal Conversion in a Muffle

The tests were performed by replacing the heating plate with a muffle with internal dimensions of 12 cm W×9.5 cm H×15 cm D, and to eliminate sample loss during conversion, 50 mL porcelain crucibles with porcelain lids were used.

The process was based on the proposal of simple operations carried out for conversion on a hot plate, but optimization led to modifications and the following was achieved: 2 mL of the solution containing a known quantity of phosphonate were transferred to a 50 mL porcelain crucible (in duplicate) and placed in the muffle, half-open to allow the escape of steam and gases during conversion.

The muffle is heated at a rate of 10° C./min⁻¹ to 450° C. and kept at this temperature for 30 minutes. After this time, the crucible is closed and removed from the muffle using long stainless steel tweezers and a glove specifically designed for working at high temperatures. For safety reasons, the closed crucible is placed in a desiccator and only when cold is the residue from the conversion taken up with 5 mL of pure water. FIG. 3 shows in detail the simple and easy-to-perform steps obtained for the process adapted for thermal conversion.

### Analytical Method Based on Digital Images

The method is physically composed of two software items (IS1 and IS2), one hardware item (IH) and one reaction item (IR), as reported in BR 102021023976-0.

Item IS1 consists of a set of regression algorithms developed with computer vision and machine learning techniques for quantifying/predicting the concentration of several analytes. Quantification is performed based on analysis with IR and processing of images of reactions originating from colorimetric images, captured by digital cameras. The regression algorithms are based on deep artificial neural networks, whose predictive models are adjusted through supervised back propagation.

Item IS2 is an application for mobile devices, developed to instruct the user/analyst and assist him/her in capturing digital images used in adjusting predictive models and in applying adjusted models in quantifying the aforementioned analytes.

The IH hardware item consists of a set of three-dimensional geometric models, called capture cameras, physically created by an additive manufacturing process (3D printing). The capture cameras are coupled to the mobile device in order to provide ideal conditions for capturing digital images subjected to predictive models.

The combination of these elements guarantees the portability of the some embodiments of the present disclosure, enabling its application in remote situations, obtaining analysis results without the need for equipment other than a cell phone.

The chemical reactions developed in the IR involve reactions with ammonium molybdate in hydrochloric acid and stannous chloride resulting in the formation and reduction of phosphomolybdic acid, which is blue in color.

The first tests were performed with samples of interest to the oil industry (Table 1). Knowing the phosphorus content determined by the ICP-OES (Inductively Coupled Plasma Atomic Emission Spectroscopy) technique, saline solutions were prepared in the range of 18 ppm of expected phosphate. The saline matrix used, called S6, has 5 molL⁻¹ of NaCl, and is consistent with the highest salinity found in production waters (pre-salt).

TABLE 1
Antiscalant agents evaluated after thermal conversion in a muffle

| | Phosphorus | 5 molL⁻¹ NaCl | |
|---|---|---|---|
| Main | concentration | PO₄³⁻ saline matrix | Solution |
| component | (mgL⁻¹) | (mgL⁻¹) | name |
| PAPEMP | 41,500 | 18.30 | A1 |
| (n = 4) | | | |
| PAPEMP | 64,900 | 18.29 | A2 |
| (n = 3) | | | |

The two solutions were subjected in duplicate to thermal conversion in a muffle. The determination of the phosphate resulting from the conversion of these products was performed by the MABID Digital Image-Based Analytical Method (Table 2).

TABLE 2
Conversion of phosphonate to orthophosphate of saline
solutions (5 molL⁻¹ of NaCl) by MABID for phosphate

| | Concentration obtained by | |
|---|---|---|
| Sample | MABID (%) (mg P/L) | Conversion |
| A1.1 | 4.15 | 69 |
| A1.2 | 4.16 | 70 |
| A1.3 | 4.77 | 80 |
| A1.4 | 4.78 | 80 |
| A1.5 | 4.66 | 78 |
| A1.6 | 4.78 | 80 |
| A2.3 | 5.38 | 90 |
| A2.4 | 5.70 | 95 |

Sample: A1 and A2 with 5.98 ppm of phosphorus in 5 molL⁻¹ NaCl solution.

What was observed in the first conversion test in the muffle:
a. the conversion of the saline solution of the PAPEMP n=4 phosphonate was around 70%;
b. the conversion of the saline solution of the PAPEMP n=3 phosphonate was around 90%; and
c. the values were considered as high conversion, compatible with the other forms of conversion mentioned in the literature, as will be seen below. Therefore, it was clear that the proposal of the inventors generates the necessary practicality of execution (FIG. 3) and the conversion results adequate for the detection of phosphonates (Table 2).

The following examples show the results comparison with the conversion method of the company HACH—commercial methodology with advanced oxidative process (Example 1); and comparison between the two conversion methodologies: phosphonate in muffle with MABID×HACH−POA/photometry (Example 2).

### Example 1—Comparison with the Conversion Method from Hach Company-commercial Methodology with Advanced Oxidative Process

The HACH spectrophotometer is a closed package, with procedures, internal calibrations and reagents already weighed and supplied in sachets.

For the conversion of a phosphonate, it presents the protocol: Method 8007 (DR 2500 photometer manual) that uses a UV lamp with increased power by a reactor and an oxidizing agent (persulfate). The lamp is sheathed with quartz which allows it to be immersed in the solution to be converted. The orthophosphate released from the phosphonate conversion is determined by spectrophotometry in the UV-Vis region using the appropriate reaction that leads to coloration. Using the internal calibration curve of the photometer, the phosphate content released is determined. The curve must be evaluated before use, with a standard provided by the system manufacturer.

The Standard Methods molybdenum blue/AcAsc protocol was evaluated by the laboratory of the inventors of the present disclosure during the development of a methodology for determining phosphate and was discarded because it did not develop color in a high salinity solution. This means that the HACH method can be based on adaptations, and it is always important to perform a preliminary evaluation to ensure that they do not result in limitations for the intended application.

The first test performed with the HACH methodology was to verify the possibility of its application to control commercial phosphonate-based antiscalant products.

### Evaluation of the Internal Curve of the DR-2800 Photometer (HACH Methodology)

a. Phosphate standard solution—10.0±0.1 mgL⁻¹—Brand: Hach
b. Prepared diluted solution 1/10 (1 mL standard solution in 10 mL volumetric flask)
c. Transferred the 10 mL to the round cell+PhosVer 3® reagent sachet (mixture of sodium molybdate, potassium pyrosulfate and ascorbic acid)
d. Blank solution with 10 mL deionized water and added the Phos Ver 3® reagent sachet (mixture of sodium molybdate, potassium pyrosulfate and ascorbic acid)
e. Result found: 9.74 mg/L PO₄³⁻ average of two determinations
f. Conclusion: for the test, the value was accepted and the curve considered good.

### Commercial Product Evaluation Using the HACH Methodology

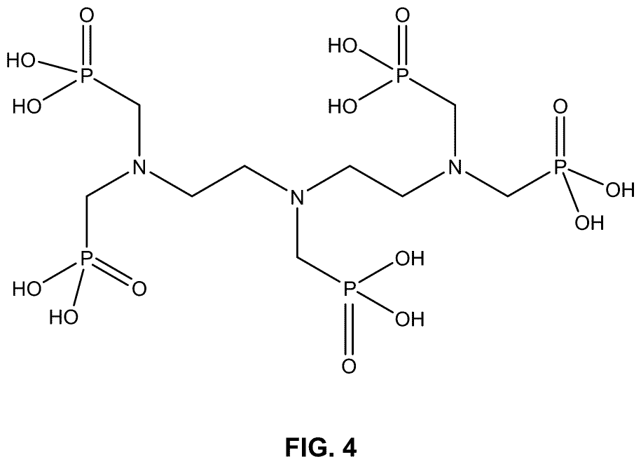

To evaluate the commercial methodology (HACH), the antiscalant DTPMP was chosen, whose molecular formula is shown in FIG. 4.

The DTPMP product containing 119,775 mgL⁻¹ of phosphonate was diluted in water in such a way as to present a concentration of 50 mgL⁻¹ of this component of the product in aqueous solution.

Following the methodology presented in Method 8007 (Table 3), the concentration of 50 mg/L of phosphonate is in the range between 0-12 5 mgL⁻¹ and, in this range, the method works the conversion with an aliquot of 1 mL of this solution in 50 mL in distilled water (S1).

TABLE 3
Expected phosphonate ranges in the
commercial sample with multipliers

| Expected range | | |
|---|---|---|
| (mg/L phosphonate) | Sample volume (mL) | Multiplier |
| 0 to 2.5 | 50 | 0.1 |
| 0 to 5 | 25 | 0.2 |
| 0 to 12.5 | 10 | 0.5 |
| 0 to 25 | 5 | 1 |
| 0 to 125 | 1 | 5 |

Source: DR 2500 Manual - Method 8007

The mixture of 25 mL of solution S1+oxidizing sachet (Potassium Persulfate for Phosphonate Powder Pillow) was subjected to the action of the UV lamp for 10 minutes; after this conversion time, 10 mL of the digested solution was transferred to the reading bottle of the DR-2800 spectrophotometer and the Phos Ver 3® reagent sachet was added. The solution containing the complex with the characteristic color of molybdenum blue was evaluated in the DR-2800 photometer; for the analytical blank, 10 mL of solution S1 (before conversion) also reacted with the Phos Ver 3® reagent sachet, thus eliminating any interference from the matrix.

The result read on the spectrophotometer in mgL⁻¹ of PO₄⁻³ is multiplied by the respective dilution factor (Multiplier) described in Table 4 and the result becomes mgL⁻¹ of phosphonate. Then, to know the concentration of the active phosphonate, the value found in mgL⁻¹ of phosphonate is multiplied by the factor shown in Table 4, relative to the DTPMP type phosphonate.

TABLE 4
Table 2 Conversion Factors by Phosphonate Type

| Phosphonate type | Conversion factor |
|---|---|
| 2-Phosphonobutane 1,2,4-Tricarboxylic | 2.84 |
| Acid (PBTC) | |
| Nitrilotrimethylphosphonic acid (NTP) | 1.05 |
| 1-Hydroxyethylidene-1,1-diphosphonic | 1.085 |
| acid (HEDPA) | |
| Ethylenediamine tetra(methylene | 1.148 |
| phosphonic acid) (EDTMPA) | |
| Hexamethylene diamine tetra (methylene | 1.295 |
| phosphonic acid) (HMDTMPA) | |
| Diethylenetriamine penta(methylene | 1.207 |
| phosphonic) acid (DETPMPA) | |
| 2-hydroxyphosphonoacetic acid (HPA) | 1.49 |

Source: DR 2500 Manual - Method 8007

The need for these factors is another limitation of the HACH method. If a given commercial product is effective, but the formula of the base phosphonate of the preparation is not known, the methodology does not indicate how to make the multiplier factor available.

Following the protocol above, the results found for the sample prepared in the laboratory containing 50 mg/L of DTPMP in deionized water are described in Table 5.

TABLE 5
Readings for solutions (deionized
water) of the reference phosphonate

| | | | Active | | | |
|---|---|---|---|---|---|---|
| Nominal | | Phos- | phos- | | | |
| DTPMP | | phonate | phonate | Average | | |
| concen- | DR2800 | concen- | concen- | active | | |
| tration | reading | tration* | tration** | phos- | | CV |
| (mgL−1) | (mgL−1) | (mgL−1) | (mgL−1) | phonate | DVP | (%) |
| 50 | 7.77 | 38.85 | 50.31 | | | |
| | 7.42 | 37.10 | 48.04 | | | |
| | 6.75 | 33.75 | 43.71 | | | |
| | 6.74 | 33.70 | 43.64 | 46.43 | 2.866 | 6.17 |

*Dilution factor: 5
**Active phosphonate factor: 1.207

The result found indicates that the HACH commercial methodology can be applied to control commercial phosphonate-based products. The average recovery of the product in the solution diluted in deionized water in the laboratory was 93% in four determinations. Taking into account that the standard is a commercial product subject to variations, the recovery was considered adequate, and it can be considered that the methodology is useful for controlling phosphonate-based products.

### Evaluation of the 8007 Methodology in Saline Solutions

The same product evaluated in distilled water was evaluated in saline solutions (synthetic seawater, synthetic formation water and in 5 molL⁻¹ saline solution). This is not intended to identify the active ingredient in a batch of the product, but rather to determine the concentration present in a process water of interest for a given operation.

The compositions of the chosen matrices are described in Table 6.

TABLE 6
Composition of the synthetic saline matrices
used for testing with the HACH methodology

| | SYNTHETIC | SYNTHETIC | |
|---|---|---|---|
| | SEAWATER | FORMATION | BRINE |
| SALTS | gL⁻¹ | WATER gL⁻¹ | gL⁻¹ |
| NaCl | 27.0622 | 91.511 | 290 |
| KCl | 0.4309 | 0.644 | — |
| MgCl₂•6H₂O | 11.7005 | 4.267 | — |
| CaCl₂•2H₂O | 1.8523 | 9.6084 | — |
| SrCl₂•6H₂O | 0.0274 | 1.1836 | — |
| Na₂SO₄ | 4.2039 | — | — |
| BaCl₂ | — | 0.402 | — |

In the same way as in aqueous solution, the product containing DTPMP (Dimethyl Triaminepentamethylene Phosphonic Acid) was subjected to conversion with UV lamp using the protocol described above. The results are indicated in Table 7.

TABLE 7
Results following the above protocol of conversion and colorimetry
for saline solutions using the DR2800 photometer

| | | | | Active | | |
|---|---|---|---|---|---|---|
| Nominal | | | Phos- | phos- | | |
| DTPMP | | | phonate | phonate | | Re- |
| concen- | | DR2800 | concen- | concen- | | cov- |
| tration | | reading | tration* | tration** | | ery |
| (mgL−1) | Diluent | (mgL−1) | (mgL−1) | (mgL−1) | DVP | (%) |
| 50 | Synthetic | 7.57 | 37.85 | 45.68 | | |
| | seawater | 6.84 | 34.20 | 41.28 | | |
| | | 8.85 | 44.25 | 53.41 | | |
| | | 6.30 | 31.50 | 38.02 | | |
| | Average | 7.39 | 36.95 | 44.60 | 5.769 | 90 |
| | Synthetic | 5.50 | 27.50 | 33.19 | | |
| | formation | 3.84 | 19.20 | 23.17 | | |
| | water | 3.98 | 19.90 | 24.02 | | |
| | | 6.08 | 30.40 | 36.69 | | |
| | Average | 4.85 | 24.25 | 29.27 | 5.814 | 58 |
| | Brine 5 | 6.42 | 32.10 | 38.74 | | |
| | molL-1 | 5.22 | 26.10 | 31.50 | | |
| | NaCl | 6.33 | 31.65 | 38.20 | | |
| | solution | 5.07 | 25.35 | 30.60 | | |
| | Average | 5.76 | 28.80 | 34.76 | 3.730 | 70 |

It was then observed that the methodology offered by HACH works very well for solutions with salinity up to seawater and falls short for solutions with increasing salinity. This situation matches the expectations that one might have from reading the manual of the manufacturer.

### Example 2—Comparison Between the Two Conversion Methodologies: Phosphonate in Muffle with MABID×HACH (POA/Photometry)

The first comparison tests between the methodologies were performed using the commercial product with the main component being DTPMP (Dimethyl Triaminepentamethylene Phosphonic Acid) diluted in brine at a concentration of 50 mgL⁻¹ of phosphonate, or 41 mgL⁻¹ of phosphate. With the same solution, 4 digestions were performed with the UV lamp (HACH) and 5 digestions in the muffle at 450° C. following the procedure already indicated in previous items. Table 8 shows the average recoveries obtained for each method and it can be concluded that the two methodologies show similar results.

TABLE 8
Conversion of the solution containing 41 mgL⁻¹ of
phosphate into brine (DTPMP) by the HACH method (UV
lamp) and by the muffle at 450° C.

| | | | RECOVERY | | | | | |
|---|---|---|---|---|---|---|---|---|
| | | (PO₄)³⁻ | HACH | | | MUFFLE | | |
| SOURCE | DILUENT | mgL⁻¹ | DR2800 | NR | SD | MABID | NR | SD |
| DTPMP | BRINE | 41 | 70 | 4 | 7.4 | 73 | 5 | 6.9 |
| (50 ppm) | | | | | | | | |

NR number of replicates SD standard deviation

In all evaluations of the conversion in a muffle, a phosphonate solution at high salinity was used. Therefore, to compare the two conversion processes, some tests were carried out with the salinity relative to a formation water. For the test, a solution containing 50 mgL⁻¹ of phosphonate DTPMP (Dimethyl Triaminepentamethylene Phosphonic Acid) in synthetic formation water, i.e. 41 ppm of phosphate, was prepared. The same conversion and detection methodologies were used (Table 9).

TABLE 9
Conversion of the solution containing 41 mgL⁻¹ of phosphate in formation
water (DTPMP) by the HACH method (UV lamp) and by muffle at 450° C.

| | | | RECOVERY | | | | | |
|---|---|---|---|---|---|---|---|---|
| | | (PO₄)³⁻ | HACH | | | MUFFLE | | |
| SOURCE | DILUENT | mgL⁻¹ | DR2800 | NR | SD | MABID | NR | SD |
| DTPMP | AF0 | 41 | 72 | 2 | 1.55 | 53 | 2 | 2.49 |
| (50 ppm) | | | | | | | | |

NR number of replicates SD standard deviation

It was considered that free phosphate and phosphonate may be present in real waters, and even some eventual decomposition of the phosphonate may be a source of phosphate, for example, if the pressure and temperature conditions lead to this. Therefore, tests were performed by mixing free phosphate with phosphonate and with this mixture the thermal conversion procedure was carried out in a muffle.

A concentration of 30 mgL⁻¹ of phosphate was maintained in the phosphonate phosphate: free phosphate ratios of 25:05, 20:10 and 15:15. DTPMT was used and, for free phosphate, KH₂PO₄ PA. Each mixture was subjected to thermal conversion in a muffle and conversion with a UV lamp (HACH), followed by the respective quantifications (MABID and photometry, respectively). Table 10 shows the duplicate results of the muffle conversion.

A free phosphate measurement was well below expectations, and it is assumed that there was some analytical error. A new solution was then prepared and digested in the muffle. Table 11 shows the conversion of the 25:05 ratio, now showing repeatability in both digestions and results for free phosphate consistent with expectations.

Table 12 shows the conversion in a UV lamp (HACH) of the same solution used to obtain the conversion in the muffle (Table 10). The results in Tables 10 and 12 compare the thermal conversion in the muffle and the conversion in a UV lamp in the presence of the HACH oxidant (potassium persulfate). The results were considered comparable.

It is a fact that the HACH method does not measure free phosphate, since it zeroes the equipment with the sample that will be subjected to conversion, nor does it indicate a solution for this, while the thermal conversion process proposed here allows this determination. Free phosphate is not distinguishable from phosphonates in other methodologies (such as ICP-OES), but the information may be relevant, since free phosphate may denote, for example, a degraded, non-active phosphonate fraction.

TABLE 10
Thermal conversion in muffle (450° C.) of phosphonate mixtures with
free phosphate at different ratios of phosphonate phosphate and free phosphate

| | | | | TOTAL | |
|---|---|---|---|---|---|
| Phosphonate | FREE | TOTAL | | PHOSPHATE | |
| phosphate:free | PHOSPHATE | PHOSPHATE | DILUITION | MINUS FREE | PHOSPHONATE |
| phosphate | MABID | MABID | FACTOR | PHOSPHATE* | CONVERSION |
| mixture (ppm) | (ppm) (ppm) | (ppm) (ppm) | 2.5 (ppm) | (ppm) | % AT (PO₄)³⁻ |
| 20:05 | 3.74* | 9.83 | 24.58 | 20.84 | 83 |
| | 5.61 | 9.93 | 24.83 | 19.22 | 77 |
| 20:10 | 10.23 | 9.76 | 24.4 | 14.17 | 71 |
| | 8.76 | 6.84 | 17.1 | 8.34 | 42 |
| | 10.25 | 10.91 | 27.28 | 17.03 | 85 |
| 15:15 | 13.95 | 8.53 | 21.33 | 7.38 | 49 |
| | 14.4 | 9.48 | 23.7 | 9.30 | 62 |

TABLE 11
Thermal conversion in muffle (450° C.) of mixtures of phosphonate
with free phosphate in the ratio of phosphonate phosphate and free
phosphate 20:05 (solution different from that evaluated in Table 10).

| | | | | TOTAL | |
|---|---|---|---|---|---|
| Phosphonate | FREE | TOTAL | | PHOSPHATE | |
| phosphate:free | PHOSPHATE | PHOSPHATE | DILUITION | MINUS FREE | PHOSPHONATE |
| phosphate | MABID | MABID | FACTOR | PHOSPHATE* | CONVERSION |
| mixture (ppm) | (ppm) (ppm) | (ppm) (ppm) | 2.5 (ppm) | (ppm) | % AT (PO₄)³⁻ |
| 20:05 | 5.62 | 9.97 | 24.93 | 19.32 | 77 |
| | 5.60 | 9.95 | 24.88 | 19.27 | 77 |

TABLE 12
Thermal conversion under UV lamp (HACH) of phosphonate mixtures with free
phosphate at different ratios of phosphonate phosphate and free phosphate

| Phosphonate | PHOSPHATE | | | |
|---|---|---|---|---|
| phosphate:free | after | DILUITION | | |
| phosphate | conversion | FACTOR | PHOSPHATE* | CONVERSION |
| mixture (ppm) | (ppm) | (ppm) | (ppm) | % AT (PO₄)³⁻ |
| 20:05 | 2.96 | 5 | 14.80 | 59 |
| | 3.27 | | 16.35 | 65 |
| 20:10 | 10.335 | 1 | 10.34 | 52 |
| | 10.51 | | 10.51 | 53 |
| 15:15 | 8.99 | 1 | 9.06 | 60 |
| | 8.92 | | 8.92 | 59 |

In conclusion, the present disclosure has great advantages over the state of the art, since a methodology has been developed that determines the presence of phosphonate in saline waters on board offshore production facilities without specific equipment and can be used with simple resources, if necessary, and the methodology for detecting phosphonates being proposed was entirely designed with the concept of a portable analytical method capable of being performed even by analysts without training in chemical operations.

It should be understood that the present description does not limit the application to the details being described and that the disclosure is capable of other embodiments and of being practiced or performed in a variety of ways, within the scope of the claims. Although specific terms have been used, such terms should be interpreted in a generic and descriptive sense, and not for the purpose of limitation.

1. A process for determining phosphonate in saline water, the process comprising the steps of:
converting the phosphonate by a thermal method, the thermal method comprising:
transferring a sample to be analyzed to a crucible with a lid, the sample comprising phosphonate;
heating the sample in the crucible with the lid half open in a muffle; and
transferring the crucible with the lid closed to a desiccator;
recovering cold residue from the converting the phosphonate step with pure water; and
determining phosphate by applying an analytical method based on digital images.

2. The process according to claim 1, wherein the NaCl concentration of the sample is equal to 5 mol·L⁻¹.

3. The process according to claim 1, wherein the crucible and lid are made of porcelain.

4. The process according to claim 1, wherein the crucible has a volume of 50 ml.

5. The process according to claim 1, wherein the muffle has internal dimensions of 12 cm W×9.5 cm H×15 cm D.

6. The process according to claim 1, further comprising heating the muffle at a rate of 10° C. min⁻¹ to a temperature of about 450° C., and maintaining the muffle at the temperature of about 450° C. for 30 minutes.

7. The process according to claim 1, wherein the sample has a volume of 2 ml and the pure water has a volume of 5 ml.
